(19) United States
(12) Patent Application Publication
(10) Pub. No.: US 2012/0129046 A1
(43) Pub. Date: May 24, 2012

(54) NONAQUEOUS SECONDARY BATTERY AND FLAME RETARDANT FOR THE SAME

(76) Inventor: Hisayuki UTSUMI, Osaka (JP)

(21) Appl. No.: 13/297,429

(22) Filed: Nov. 16, 2011

(30) Foreign Application Priority Data
Nov. 19, 2010 (JP) 2010-259161

(51) Int. Cl. H01M 10/0564; C07D 263/44; C07D 263/22

(52) U.S. Cl. 429/199; 429/188; 548/229; 548/226

(57) ABSTRACT

A nonaqueous secondary battery, comprising: a positive electrode; a negative electrode; and a nonaqueous electrolyte solution, the nonaqueous electrolyte solution containing at least a cyclic compound having, in the molecule, a functional group having an ester bond to which a nitrogen atom is attached, in which is the general formula (I).

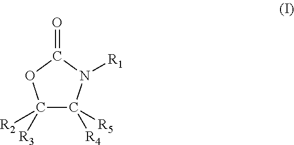

### CROSS-REFERENCE TO RELATED APPLICATION

This application is related to Japanese application No. 2010-259161 filed on Nov. 19, 2010, whose priority is claimed under 35 USC §119, the disclosure of which is incorporated by reference in its entirety.

### BACKGROUND OF THE INVENTION

1. Field of the Invention

The present invention relates to a nonaqueous secondary battery and a flame retardant for the battery. More particularly, the present invention relates to a nonaqueous secondary battery that has battery performance comparable to conventional batteries and that is superior in safety to conventional batteries, and to a flame retardant for the nonaqueous secondary battery.

2. Description of the Related Art

In recent years, reduction in size and weight of electronic devices has been remarkably progressed, and with the progress, it has been demanded that secondary batteries that are used for such electronic devices should have higher energy density. An example of secondary batteries that can meet the demand is a secondary battery including a nonaqueous electrolyte solution (hereinafter, referred to as nonaqueous secondary battery) such as a lithium-ion secondary battery.

In the lithium-ion secondary battery, a nonaqueous electrolyte solution is used, and the nonaqueous electrolyte solution comprises an electrolyte salt such as a lithium salt and a nonaqueous solvent. The nonaqueous solvent is desired to have high dielectric constant and high oxidation potential, and to be stable in batteries regardless of operation environment.

As such a nonaqueous solvent, aprotic solvents are used, and known examples thereof include high-permittivity solvents such as cyclic carbonates including ethylene carbonate and propylene carbonate, and cyclic carboxylate esters including γ-butyrolactone; and low-viscosity solvents such as chain carbonates including diethyl carbonate and dimethyl carbonate, and ethers including dimethoxyethane. Usually, a high-permittivity solvent and a low-viscosity solvent are used in combination.

However, the lithium-ion secondary battery including a nonaqueous electrolyte solution may suffer from leakage of the nonaqueous electrolyte solution due to a defect involving increased internal pressure caused by breakage of the battery or any other reason. The leakage of the nonaqueous electrolyte solution may lead to short-circuit between a positive electrode and a negative electrode constituting the lithium-ion secondary battery to cause generation of fire or burning. It may also lead to generation of heat in the lithium-ion secondary battery to cause vaporization and/or decomposition of the organic solvent-based nonaqueous solvent to produce gas. In some cases, the produced gas caught fire or caused rupture of the lithium-ion secondary battery.

In order to solve the above-described problems, studies have been carried out to give flame retardancy by adding a flame retardant to the nonaqueous electrolyte solution.

Techniques to add a flame retardant to a nonaqueous electrolyte solution is proposed in Japanese Unexamined Patent Publication No. 2001-338682, Japanese Unexamined Patent Publication (Translation of PCT Application) No. 2001-525597 and Japanese Unexamined Patent Publication No. HEI 11(1999)-329495, for example.

As the flame retardant, specifically, Japanese Unexamined Patent Publication No. 2001-338682 proposes phosphazene derivatives, Japanese Unexamined Patent Publication (Translation of PCT Application) No. 2001-525597 proposes azobis (isobutyronitrile) (AIBN), and Japanese Unexamined Patent Publication No, HEI 11(1999)-329495 proposes imidazole compounds.

### SUMMARY OF THE INVENTION

According to an aspect of the present invention, therefore, there is provided a nonaqueous secondary battery, comprising: a positive electrode; a negative electrode; and a nonaqueous electrolyte solution, the nonaqueous electrolyte solution containing at least a cyclic compound having, in the molecule, a functional group having an ester bond to which a nitrogen atom is attached, in which is the general formula (I):

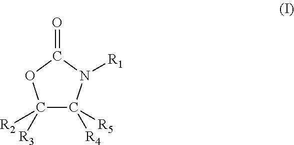

wherein R₁ is a hydrogen atom or, a group selected from lower alkyl group, lower alkenyl group, lower alkoxy group, lower alkoxycarbonyl group, lower alkylcarbonyl group, lower cycloalkyl group and aryl group that may be have a substituent;

R₂ and R₃ may be the same or different and each represents a halogen atom or, a group selected from lower alkyl group, lower alkenyl group, lower alkoxy group, lower alkoxycarbonyl group, lower alkylcarbonyl group, lower cycloalkyl group and aryl group that may be have a substituent or R₂ and R₃ may be combined to form ═CH₂ or ═O; and

R₄ and R₅ may be the same or different and each represents a hydrogen atom, a halogen atom or, a group selected from lower alkyl group, lower alkenyl group, lower alkoxy group, lower alkoxycarbonyl group, lower alkylcarbonyl group, lower cycloalkyl group and aryl group that may be have a substituent or R₄ and R₅ may be combined to form ═CH₂ or ═O.

According to another aspect of the present invention, there is provided a flame retardant for a nonaqueous secondary battery, comprising a cyclic compound of the general formula (I) having, in the molecule, a functional group having an ester bond to which a nitrogen atom.

These and other objects of the present application will become more readily apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF THE PREFERRED EMBODIMENTS

While producing excellent flame retardancy, phosphazene derivatives are expected to cause unstable operation of the lithium-ion secondary battery when used with certain kinds of nonaqueous solvents or blended with a nonaqueous solvent at certain blending ratios, and when used in a certain temperature environment, in particular, at high temperature. Generally, when the lithium-ion secondary battery generates heat for some reasons, thermal decomposition reaction occurs at an interface between a negative electrode or a positive electrode and the electrolyte solution, and in the case of thermal runaway of this reaction, the lithium-ion secondary battery may be ruptured or catch fire. This phenomenon can occur even when a phosphazene derivative is blended. In addition, since the phosphazene derivative becomes a membrane on the surface of the negative electrode, battery characteristics such as cycle characteristics and environmental stability in operation may be degraded.

In an Example in Japanese Unexamined Patent Publication No. 2001-338682, a phosphazene derivative is used at a high content of 40% by volume with respect to a nonaqueous solvent. Since the phosphazene derivative has relatively high viscosity and relatively low dielectric constant, operation of a battery having a high phosphazene derivative content in a low-temperature environment causes concern about reduction in the electric conductivity of the nonaqueous electrolyte solution and degradation in the battery performance due to the reduction,

Meanwhile, AIBN is less soluble in nonaqueous solvents typified by aprotic solvents, and therefore the content thereof cannot be increased. Accordingly, AIBN may not improve flame retardancy sufficiently. Furthermore, AIBN may be electrolyzed due to charge and discharge of the lithium-ion secondary battery, causing concern about degradation in battery performance.

Likewise, imidazole compounds do not produce sufficient flame retardancy unless the content thereof is increased. However, an increased content thereof causes concern about degradation in the cycle characteristics and the environmental stability in operation.

It is therefore desired to further improve flame retardancy without degrading battery performance.

The inventor of the present invention has made intensive studies about flame retardants for nonaqueous secondary batteries and, as a result, unexpectedly found that a battery is enabled to produce sufficient flame retardancy when a nonaqueous electrolyte solution therein contains a “cyclic compound having, in the molecule, a functional group having an ester bond to which a nitrogen atom is attached”, to achieve the present invention. As a result of the sufficient flame retardancy thus produced, safety and reliability of the nonaqueous secondary battery can be ensured even when the battery is abnormally heated. Furthermore, this flame retardant does not affect electric characteristics of the nonaqueous secondary battery over a wide temperature range to allow provision of a nonaqueous secondary battery showing stable cycle characteristics.

A nonaqueous secondary battery of the present invention comprises: a positive electrode; a negative electrode; and a nonaqueous electrolyte solution, and the nonaqueous electrolyte solution contains at least a compound having a structure represented by the general formula (I).

The inventor believes that the mechanism for the “cyclic compound having, in the molecule, a functional group having an ester bond to which a nitrogen atom is attached” used in the present invention as a flame retardant to exert flame retardancy is as follows: in the case of thermal runaway, which starts fire, of the nonaqueous secondary battery, thermal decomposition is caused to generate inert gas containing CO₂ or CO as a main component and, as a result, reduce the ambient oxygen concentration thereby to extinguish the fire (anoxic extinction). In order to achieve such a mechanism, it is essential that the compound of the present invention has the “functional group having an ester bond to which a nitrogen atom is attached” in the molecule of the cyclic structure

(1) Compound Represented by General Formula (I)

Hereinafter, the compound of the general formula (I), that is, the “cyclic compound having, in the molecule, a functional group having an ester bond to which a nitrogen atom is attached” will be also referred to as “compound of the present invention”.

The compound of the present invention is represented by the general formula (I):

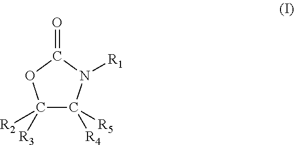

R₁ is a hydrogen atom or, a group selected from lower alkyl group, lower alkenyl group, lower alkoxy group, lower alkoxycarbonyl group, lower alkylcarbonyl group, lower cycloalkyl group and aryl group that may have a substituent.

In the present invention, the term “lower” means 1 to 6 carbon atoms. In the case of the cycloalkyl group, however, the term “lower” means 3 to 6 carbon atoms.

As the lower alkyl group, may be mentioned linear or branched alkyl group having 1 to 6 carbon atoms. Specific examples thereof include methyl group, ethyl group, n-propyl group, isopropyl group, n-butyl group, isobutyl group, sec-butyl group, tert-butyl group, n-pentyl group, isopentyl group, neopentyl group, tert-pentyl group, n-hexyl group and isohexyl group. Out of them, alkyl group having 1 to 4 carbon atoms is preferable, and methyl group and tert-butyl group are particularly preferable.

As the lower alkenyl group, may be mentioned linear or branched alkenyl group having 1 to 6 carbon atoms, and alkenyl group having 1 to 4 carbon atoms is preferable. Specific examples thereof include vinyl group, 1-propenyl group, allyl group (2-propenyl group), -butenyl group, 2-butenyl group and 3-butenyl group. Out of them, vinyl group is particularly desirable.

As the lower alkoxy group, may be mentioned linear or branched alkoxy group having 1 to 6 carbon atoms. Specific examples thereof include methoxy group, ethoxy group, n-propoxy group, isopropoxy group, n-butoxy group, isobutoxy group, sec-butoxy group, tert-butoxy group, n-pentyloxy group, isopentyloxy group, neopentyloxy group, tert-pentyloxy group, n-hexyloxy group and isohexyloxy group. Out of them, alkoxy group having 1 to 4 carbon atoms is preferable, and methoxy group is particularly preferable.

The lower alkoxycarbonyl group is group which is derived from a lower fatty acid and in which an alcohol residue is removed. Specific examples thereof include formyloxy group, acetoxy group, propionyloxy group, butyryloxy group, isobutyryloxy group, valeryloxy group, isovaleryloxy group and pivaloyloxy group. Out of them, alkoxycarbonyl group having 1 to 4 carbon atoms is preferable, and acetoxy group is particularly preferable.

The lower alkylcarbonyl group is acyl group derived from a lower fatty acid, that is, lower fatty acyl group. Specific examples thereof include formyl group, acetyl group, propionyl group, butyryl group, isobutyryl group, valeryl group, isovaleryl group and pivaloyl group. Out of them, alkylcarbonyl group having 1 to 4 carbon atoms is preferable, and acetyl group is particularly preferable.

As the lower cycloalkyl group, may be mentioned cycloalkyl group having 3 to 6 carbon atoms. Specific examples thereof include cyclopropyl group, cyclobutyl group, cyclopentyl group and cyclohexyl group. Out of them, cycloalkyl group having 3 or 4 carbon atoms is preferable, and cyclopropyl group and cyclobutyl group are particularly preferable.

As the aryl group, may be mentioned aryl group having 6 to 10 carbon atoms. Specific examples thereof include phenyl group, 1-naphthyl group and 2-naphthyl group. Out of them, phenyl group and 2-naphthyl group are particularly preferable.

Examples of the substituent that R₁ may have include halogen atoms such as a fluorine atom, a chlorine atom and a bromine atom; lower alkyl group as described above; lower alkoxy group as described above; aryl group as described above; and aryloxy group.

Examples of the optionally substituted group include p-tolyl group.

R₂ and R₃ may be the same or different and each represents a halogen atom or, a group selected from lower alkyl group, lower alkenyl group, lower alkoxy group, lower alkoxycarbonyl group, lower alkylcarbonyl group, lower cycloalkyl group and aryl group that may have a substituent.

Examples of the lower alkyl group, the lower alkenyl group, the lower alkoxy group, the lower alkoxycarbonyl group, the lower alkylcarbonyl group, the lower cycloalkyl group and the aryl group to be selected as R₂ and R₃, and the substituent that R₃ may have include those mentioned for R₁.

Examples of the halogen atom include a fluorine atom, a chlorine atom and a bromine atom. Out of them, a chlorine atom and a fluorine atom are preferable, and a chlorine atom is particularly preferable.

R₂ and R₃ may be combined ═CH2 or ═O.

R₄ and R₅ may be the same or different and each represents a hydrogen atom, a halogen atom or, a group selected from lower alkyl group, lower alkenyl group, lower alkoxy group, lower alkoxycarbonyl group, lower alkylcarbonyl group, lower cycloalkyl group and aryl group that may have a substituent. Any of the halogen atoms and the groups mentioned for R₂ and R₃ can be used as the halogen atoms and the groups usable as R₄ and R₅.

The solubility of the compound of the present invention in an aprotic solvent can be controlled by controlling the kinds of the substituents R₁ to R₅, for example. Accordingly, the compound of the present invention is enabled to have no effect on the electric characteristics of the nonaqueous secondary battery in a normal situation and to decompose to produce inert gas containing CO or CO as a main component thereby to control thermal runaway in an abnormal situation. The solubility can be increased by increasing the number of carbon atoms of R₁ to R₅ or using an aromatic group, for example.

Preferably, the compound of the present invention is a compound that produces inert gas containing CO₂ or CO as a main component when heated at a temperature higher than its decomposition temperature. The decomposition temperature is preferably 100° C. or more higher than normal ambient temperature where the nonaqueous secondary battery is used. Specifically, the decomposition temperature is preferably 100° C. to 300° C., and more preferably 120° C. to 250° C., and still more preferably 140° C. to 250° C. When the difference between the decomposition temperature and the normal ambient temperature is less than 100° C., the compound of the present invention may decompose during normal use, and in this case, the electric characteristics of the nonaqueous secondary battery will be degraded. Here, the decomposition temperature can be controlled by controlling substituent effects.

Examples of the compound of the general formula (I) for providing the above-described production of inert gas and decomposition temperature range include a compound formed of a combination of R₁ selected from a hydrogen atom and lower alkyl group, R₂ and R₃ selected from lower alkyl group, and R₄ and R₅ selected from a hydrogen atom and ═O when combined together.

Specific examples of the compound include
5,5-dimethyl-1,3-oxazolidine-2-one,
4,4,5,5-trimethyl-1,3-oxazolidine-2-one,
3,5,5-trimethyloxazolidine-2,4-dione,
   5,5-dimethyl-3-ethyl-2,4-oxazolidinone,
   5,5-dimethyl-3-methyl-2,4-oxazolidinone,
   5,5-diethyl-3-methyl-2,4-oxazolidinone and
   3,5,5-triethyl-2,4-oxazolidinone.

The compound of the present invention can be produced by commonly known methods or may be commercially available products as described in Examples.

As the compound of the formula (I), an objective substance can be obtained by reacting amino alcohol and cyclic carbonate, and then bringing the reaction product into contact with cation-exchange resin, for example. Alternatively, an objective substance can be obtained by reacting amino alcohol and cyclic carbonate in the presence of a catalyst (acid or base adsorbent).

(2) Nonaqueous Electrolyte Solution

The nonaqueous electrolyte solution contains an electrolyte salt, a nonaqueous solvent and, optionally, an additive. The compound of the present invention can function as a nonaqueous solvent. When the compound of the present invention by itself can provide a nonaqueous electrolyte solution having sufficient properties, therefore, no additional organic solvent needs to be used. However, in terms of enhancement in charge/discharge characteristics and resistance to low temperature of the nonaqueous secondary battery, the nonaqueous solvent is preferably a mixed solvent with an additional organic solvent.

As the additional organic solvent, aprotic organic solvents can be usually used. Examples of the aprotic organic solvents include, but not particularly limited to, dimethyl carbonate, diethyl carbonate, ethyl methyl carbonate, dipropyl carbonate, propylene carbonate, ethylene carbonate, butylene carbonate, γ-butvrolactone, γ-valerolactone, tetrahydrofuran, 2-methyltetrahydrofuran, dimethyl sulfoxide, 1,3-dioxolane, formamide, dimethylformamide, acetonitrile, ruethyl formate, methyl acetate, diethyl ether, 1,2-dimethoxyethane, 1,2-diethoxyethane, ethoxymethoxyethane, dioxane, sulfolane and methylsulfolane. One or more kinds of these organic solvents may be used independently or in combination.

The percentage of the compound of the present invention to be blended in the nonaqueous electrolyte solution is usually in a range of 1% to 60% (v/v), and preferably in a range of 10% to 40% by volume fraction. When the percentage is less than 1%, rupture and generation of fire of the nonaqueous secondary battery may not be sufficiently inhibited. On the other hand, when the percentage is more than 60%, the performance of the nonaqueous secondary battery may be deteriorated in a low-temperature environment.

As the electrolyte salt, a lithium salt is usually used. The lithium salt is not particularly limited, as long as it dissolves in the nonaqueous solvent. Examples thereof include LiClO₄, LiCl, LiBF₄, LiPF₆, LiAsF₆, LiSbF₆, LiN(SO₂CF₃)₂, LiC(SOCF₃)₂, lower aliphatic carboxylic acid, chioroborane lithium and lithium tetraphenyborate. One or more kinds of these lithium salts can be used independently or in combination. The amount of the electrolyte salt to add is preferably 0.1 mol to 3 mol, and more preferably 0.5 mol to 2 mol with respect to 1 kg of the nonaqueous solvent.

Examples of the additive include conventionally known dehydrators and deoxidizers. Specific examples thereof include vinylene carbonate, fluoroethylene carbonate, trifluoropropylene carbonate, phenyl ethylene carbonate, succinic anhydride, glutaric anhydride, maleic anhydride, ethylene sulfite, 1,3-propane sultone, 1,4-butane sultone, methyl methanesulphonate, dibutylsulphide, heptane, octane and cycloheptarie. When they are usually contained in the nonaqueous solvent at a concentration of 0.1% by weight or more to 5% by weight or less, the capacity retention characteristics and the cycle characteristics after storage in a high-temperature environment can be improved.

(3) Positive Electrode

The positive electrode can be produced by applying, drying and pressurizing a paste containing, for example, a positive-electrode active material, a conductive material, a binder and an organic solvent on a positive-electrode current collector. The conductive material in an amount of 1 part by weight to 20 parts by weight, the binder in an amount of I part by weight to 15 parts by weight and the organic solvent in an amount of 30 parts by weight to 60 parts by weight can be blended with respect to 100 parts by weight of the positive-electrode active material.

Examples of the positive-electrode active material usable here include lithium complex oxides such as LiNiO₂, LiCoO₂ and LiMn₂O₄; and compounds obtained by substituting one or more elements in these oxides with other elements (far example, Fe, Si, Mo, Cu and Zn).

Examples of the conductive material include carbonaceous materials such as acetylene black and ketjen black.

Examples of the binder include polyvinylidene fluoride (PVdF), polyvinyl pyridine and polytetrafluoroethylene.

Examples of the organic solvent include N-methyl-2-pyrrolidone (NMP) and N,N-dimethylformamide (DMF).

Examples of the positive-electrode current collector include a foil or a thin sheet of a conductive metal such as SUS and aluminum.

(4) Negative Electrode

The negative electrode can be produced by applying, drying and pressurizing a paste containing, for example, a negative-electrode active material, a conductive material, a binder and an organic solvent on a negative-electrode current collector. The conductive material in an amount of 1 part by weight to 15 parts by weight, the binder in an amount of 1 part by weight to 10 parts by weight and the organic solvent in an amount of 40 parts by weight to 70 parts by weight can be blended with respect to 100 parts by weight of the negative-electrode active material.

Examples of the negative-electrode active material include pyrolyzed carbons, cokes, graphites, vitreous carbons, sintered body of organic polymer compounds, carbon fibers and activated carbons.

Examples of the conductive material include carbonaceous materials such as acetylene black, ketjen black and vapor grown carbon fiber (VGCF).

Examples of the binder include polyvinylidene fluoride, polyvinyl pyridine and polytetrafluoroethylene.

Examples of the organic solvent include N-methyl-2-pyrrolidone (NMP) and N,N-dimethylformamide (DMF).

Examples of the negative-electrode current collector include a foil of a metal such as copper.

Usually, a separator is interposed between the negative electrode and the positive electrode.

(5) Others

A separator may be interposed between the negative electrode and the positive electrode. The material of the separator is usually a porous film, and can be selected in view of solvent resistance and reducibility resistance. Suitable examples thereof include a porous film and a nonwoven fabric of polyolefin resin such as polyethylene and polypropylene. The film and the nonwoven fabric of such materials may be used as a single layer or multiple layers. In the case of multiple layers, it is preferable that at least one sheet of a nonwoven fabric is used in view of the cycle characteristics, performance at low temperature and load characteristics.

The separator is optionally interposed between the negative electrode and the positive electrode, and then a nonaqueous electrolyte solution is injected thereto to obtain a nonaqueous secondary battery. In addition, this nonaqueous secondary battery, as a unit, may be stacked into multiple layers.

Other than those mentioned, generally used and commonly known members can be used to constitute the nonaqueous secondary battery (for example, current collector).

In addition, the form of the nonaqueous secondary battery is not particularly limited, and examples thereof include various forms such as a button type, a coin type, a rectangular type, a cylinder type having a spiral structure and a laminate type, which can be varied in size such as a thin type and a large size according to use.

### EXAMPLE

Hereinafter, the present invention will be described in detail with reference to examples and comparative examples; however, the present invention is not limited to the following examples and comparative examples at all.

### Example 1

To 80 ml of a mixed solvent of ethylene carbonate and diethylene carbonate (mixing ratio (volume ratio): ethylene carbonate/diethylene carbonate=1/2) (aprotic organic solvent), ml of 5,5-dimethyl-1,3-oxazolidin-2-one (product by Sigma-Aldrich Co., shown as “A” in Table 1) represented by the following formula was added. In the resulting mixed solution, LiPF6 as a lithium salt was dissolved at a concentration of 1.0 mol/kg to prepare a nonaqueous electrolyte solution.

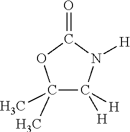

LiMn₂O₄ as a positive-electrode active material in an amount of 100 parts by weight, acetylene black as a conductive material in an amount of 5 parts by weight, PVdF as a binder in an amount of 5 parts by weight and NMP as a solvent in an amount of 40 parts by weight were kneaded for dispersion with a planetary mixer to prepare a paste for positive electrode formation. The paste prepared was applied with a coater to uniformly coat both sides of a band-like aluminum foil having a thickness of 20 μm constituting a positive-electrode current collector. Here, an end portion of the aluminum foil was left uncoated for connection of a terminal. The coat was dried under vacuum at 130° C. for 8 hours to remove the solvent, and then pressed by using a hydraulic press machine to form a positive plate. The positive plate obtained was cut into a predetermined size for use

A natural powdered graphite manufactured in China as a negative-electrode active material (average particle diameter: 15 μm) in an amount of 100 parts by weight, VGCF powder (VGCF, high-bulk-density product by Showa Denko K.K.) as a conductive material in an amount of 2 parts by weight, PVdF as a binder in an amount of 2 parts by weight and NMP as a solvent in an amount of 50 parts by weight were kneaded for dispersion with a planetary mixer to prepare a paste for negative electrode formation. The paste prepared was applied with a coater to uniformly coat both sides of a copper foil having a thickness of 10 μm constituting a negative-electrode current collector. Here, an end portion of the copper foil was left uncoated for connection of a terminal. Further, the coat was dried under vacuum at 100° C. for 8 hours to remove the solvent, and then pressed by using a hydraulic press machine to form a negative plate. The negative plate obtained was cut into a predetermined size for use.

The positive and negative plates obtained were stacked to form a laminate with a polypropylene porous film as a separator interposed therebetween, and then the nonaqueous electrolyte solution was injected into the laminate to produce a nonaqueous secondary battery.

### Example 2

A nonaqueous secondary battery was produced in the same manner as in Example 1 except that the amount of the mixed solvent of ethylene carbonate and diethylene carbonate was changed to 99 ml, and the amount of the 5,5-dimethyl-1,3-oxazolidin-2-one (product by Sigma-Aldrich Co.) was changed to 1 ml.

### Example 3

A nonaqueous secondary battery was produced in the same manner as in Example 1 except that the amount of the mixed solvent of ethylene carbonate and diethylene carbonate was changed to 40 ml, and the amount of the 5,5-dimethyl-1,3-oxazolidine-2-one (product by Sigma-Aldrich Co.) was changed to 60 ml.

### Example 4

A nonaqueous secondary battery was produced in the same manner as in Example 1 except that the amount of the mixed solvent of ethylene carbonate and diethylene carbonate was changed to 95 ml, and 5 ml of 3,5,5-trimethyloxazolidine-2,4-dione (product by Sigma-Aldrich Co., shown as “3” in Table 1) represented by the following formula was used as the compound of the present invention.

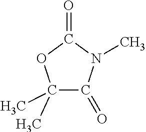

### Example 5

A nonaqueous secondary battery was produced in the same manner as in Example 1 except that the amount of the mixed solvent of ethylene carbonate and diethylene carbonate was changed to 95 ml, and 5 ml of 5,5-dimethyl-3-ethyl-2,4-oxazolidinone (product by Sigma-Aldrich Co., shown as “C” in Table 1) represented by the following formula was used.

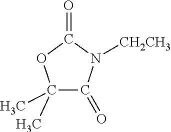

### Comparative Example 1

A nonaqueous secondary battery was produced in the same manner as in Example 1 except that no compound of the present invention was used.

### Comparative Example 2

A nonaqueous secondary battery was produced in the 1.0 same manner as in Example 1 except that the amount of the mixed solvent of ethylene carbonate and diethylene carbonate was changed to 98 parts by weight, and 2 parts by weight of AIBN (azobisisobutyronitrile, product by Tokyo Chemical Industry Co., Ltd.) was added to the mixed solvent instead of the compound of the present invention to prepare and use 100 ml of a mixed solution.

Method for Testing Battery Performance

The nonaqueous secondary batteries obtained in Examples 1 to 5, and Comparative Examples 1 and 2 were measured for the initial discharge capacity and the discharge capacity retention at 20° C. and 60° C., and tested for the safety by a nail penetration test as follows.

(1) Measurement for Initial Discharge Capacity at 20° C.

The capacity measured after each nonaqueous secondary battery is charged up to 4.2 V at a rate of 0.1 CmA, and then discharged down to 3.0 V at a rate of 0.1 CmA is determined as the initial discharge capacity (mAh/g). The measurement is performed in an incubator set to a constant temperature of 20° C.

(2) Measurement for Discharge Capacity Retention at 20° C.

A cycle of charging each nonaqueous secondary battery up to 4.2 V at a rate of 1 CmA and discharging the battery down to 3.0 V at a rate of 1 CmA is repeated 99 times, and then a cycle of charging and discharging under the same condition as in the measurement for the initial discharge capacity is completed for the 100th time in total, whereupon the battery is measured for the capacity.

After completion of the measurement for the 100th time, a cycle of charging each nonaqueous secondary battery up to 4.2 V at a :rate of 1 CmA and discharging the battery down to 3.0 V at a rate of 1 CmA is repeated 499 times, and then a cycle of charging and discharging under the same condition as in the measurement for the initial discharge capacity is completed for the 500th time in total, whereupon the battery is measured for the capacity.

The discharge capacity retention (%) at the 100th cycle and the discharge capacity retention (%) at the 500th c_{y}cle are defined as the percentage of the initial discharge capacity accounted for by the discharge capacity at the 100th cycle and the percentage of the initial discharge capacity accounted for by the discharge capacity at the 500th cycle, respectively. The measurement is performed in an incubator set to a constant temperature of 20° C.

(3) Initial Discharge Capacity and Discharge Capacity Retention at 60° C.

The measurement for the initial discharge capacity (mAh/g) and the measurement for the discharge capacity retention (%) at 60° C. are performed in the same manner as in the measurement for the initial discharge capacity and the measurement for the discharge capacity retention at 20° C. except that the temperature of the incubator is set to a constant temperature of 60° C.

(4) Nail Penetration Test

As the nail penetration test, each nonaqueous secondary battery are charged up to 4.2 V at a rate of 0.1 CmA, and then the nail having a diameter of 3 mm penetrates the battery at a speed of 1 mm/s at a room temperature of 20° C. to observe the state of the battery.

Table 1 shows test results together with the constituent materials of the nonaqueous electrolyte solutions and their percentages.

The abbreviations in Table 1 represent the followings:

LiPF6: lithium salt LiPF₆

EC/DEC: mixed solvent of ethylene carbonate and diethylene carbonate

A: 5,5-dimethyl-1,3-oxazolidin-2-one

B: 3,5,5-trimethyloxazolidine-2,4-dione

C: 5,5-dimethyl-3-ethyl-2,4-oxazolidinone

AIBN: azobisisobutyronitrile

TABLE 1

| | | | Example | | | | | Com. Ex. | |
|---|---|---|---|---|---|---|---|---|---|
| | | | 1 | 2 | 3 | 4 | 5 | 1 | 2 |
| Nonaqueous | Electrolyte | Kind | LiPF6 | LiPF6 | LiPF6 | LiPF6 | LiPF6 | LiPF6 | LiPF6 |
| electrolyte | salt | | | | | | | | |
| solution | Nonaqueous | Kind | EC/DEC | EC/DEC | EC/DEC | EC/DEC | EC/DEC | EC/DEC | EC/DEC |
| | solvent | Volume ratio | 1/2 | 1/2 | 1/2 | 1/2 | 1/2 | 1/2 | 1/2 |
| | | Percentage | 80 (V/V %) | 99 (V/V %) | 40 (V/V %) | 95 (V/V %) | 95 (V/V %) | 100 (V/V %) | 98 pbw |
| | | | | | | | | | (98 wt %) |
| | Cyclic | Kind | A | A | A | B | C | — | AIBN |
| | compound | Percentage | 20 (V/V %) | 1 (V/V %) | 60 (V/V %) | 5 (V/V %) | 5 (V/V %) | — | 2 pbw |
| | | | | | | | | | (2 wt %) |
| Electric | Initial | Dis. capa. | 117.9 | 120.4 | 116.2 | 118.1 | 118.4 | 115.3 | 91.2 |
| characteristics | 100^{th} | Dis. capa. | 113.2 | 118.0 | 109.2 | 112.2 | 113.7 | 106.1 | 82.1 |
| (20° C.) | cycle | Dis. capa. ret. | 96 | 98 | 94 | 95 | 96 | 92 | 90 |
| | 500^{th} | Dis. capa. | 104.9 | 110.8 | 101.1 | 101.6 | 103.0 | 94.1 | 68.4 |
| | cycle | Dis. capa. ret. | 89 | 92 | 87 | 86 | 87 | 82 | 75 |
| Electric | Initial | Dis. capa. | 118.1 | 119.1 | 116.1 | 117.1 | 117.7 | 112.6 | — |
| characteristics | 100^{th} | Dis. capa. | 106.3 | 110.8 | 99.8 | 105.4 | 108.3 | 89.0 | — |
| (60° C) | cycle | Dis. capa. ret. | 90 | 93 | 86 | 90 | 92 | 79 | — |
| | 500^{th} | Dis. capa. | 94.5 | 100.0 | 88.2 | 92.5 | 100.0 | 68.0 | — |
| | cycle | Dis. capa. ret. | 80 | 84 | 76 | 79 | 85 | 61 | — |

| | | | | | | | |
|---|---|---|---|---|---|---|---|
| Nail penetration test | Nae | Nae | Nae | Nae | Nae | SF | SF |

Com. Ex. = Comparative Example pbw = parts by weight Dis. capa. = Discharge capacity (mAh/g) Dis. capa. ret. = Discharge capacity retention (%) Nae = No abnormal event SF = Smoke Fire

The results shown in Table 1 have revealed the followings:

The general nonaqueous secondary battery including a general organic solvent as a nonaqueous solvent and containing no flame retardant (Comparative Example 1) experienced generation of smoke and generation of fire in the nail penetration test. The nonaqueous secondary battery containing AIBN, which is a general flame retardant, (Comparative Example 2) also experienced generation of smoke and generation of fire in the nail penetration test as in the case of Comparative Example 1.

On the other hand, the nonaqueous secondary batteries in which the nonaqueous solvent contains a compound of the present invention (Examples 1 to 5) did not experience abnormal events such as generation of smoke and generation of fire in the nail penetration test. Furthermore, in terms of the battery performance, the nonaqueous secondary batteries of Examples 1 to 5 produced significantly good results compared with the nonaqueous secondary battery of Comparative Example 2 containing AIBN, which is a general flame retardant.

In addition, the nonaqueous secondary battery of Comparative Example 2 experienced electrolysis of AIBN in the electrolyte solution during the charging and the discharging at 20° C. and 60 ° C., showed deterioration of the cycle characteristic at 20° C., and failed to provide stable electrochemical characteristics at 60° C.

As described above, Table 1 indicates that use of a compound of the present invention as a flame retardant in a nonaqueous electrolyte solution enables production of a nonaqueous secondary battery improved in the flame retardancy and comparable in the electric characteristics to conventional batteries.

In the present invention, a nonaqueous secondary battery is enabled to produce sufficient flame retardancy by including a “cyclic compound having, in the molecule, a functional group having an ester bond to which a nitrogen atom is attached” in a nonaqueous electrolyte solution. As a result, risk of thermal runaway can be reduced even in an abnormal situation such as where the internal temperature of the nonaqueous secondary battery rises due to short-circuit, overcharge or any other reasons. In addition, this cyclic compound has less impact on electric characteristics of the nonaqueous secondary battery including cycle characteristics. Accordingly, it is possible to provide a nonaqueous secondary battery improved in safety and reliability.

When the compound of the general formula (I) is contained in the nonaqueous electrolyte solution at a proportion of 1% by volume to 60% by volume, it is possible to provide a nonaqueous secondary battery more improved in safety and reliability.

When the compound of the general formula (I) is a compound that produces inert gas containing CO₂ or CO as a main component when heated at a temperature higher than its decomposition temperature, it is possible to provide a nonaqueous secondary battery more improved in safety and reliability.

When the compound of the general formula (I) is a compound having a decomposition temperature of 120° C. to 250° C., it is possible to provide a nonaqueous secondary battery more improved in safety and reliability.

When the lower alkyl group, the lower alkenyl group and the lower alkoxy group are alkyl, alkenyl and alkoxy having 1 to 6 carbon atoms, and the lower cycloalkyl group is cycloalkyl having 3 to 6 carbon atoms, it is possible to provide a nonaqueous secondary battery more improved in safety and reliability,

When the compound of the general formula (I) is a compound formed of a combination of R₁ selected from a hydrogen atom and lower alkyl group, R₂ and R₃ selected from lower alkyl group, and R₄ and R₅ selected from a hydrogen atom and ═O combined, it is possible to provide a nonaqueous secondary battery more improved in safety and reliability.

Furthermore, because of the above-described effects, it is possible to provide a flame retardant, for a nonaqueous secondary battery, being capable of improving the safety and the reliability of the nonaqueous secondary battery.

What is claimed is:

1. A nonaqueous secondary battery, comprising: a positive electrode; a negative electrode and a nonaqueous electrolyte solution, the nonaqueous electrolyte solution containing at least a cyclic compound having, in the molecule, a functional group having an ester bond to which a nitrogen atom is attached, in which is the general formula (I): 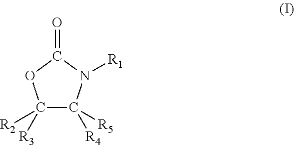
wherein R₁ is a hydrogen atom or, a group selected from lower alkyl group, lower alkenyl group, lower alkoxy group, lower alkoxycarbonyl group, lower alkylcarbonyl group, lower cycloalkyl group and aryl group that may be have a substituent;
R₂ and R₃ may be the same or different and each represents a halogen atom or, a group selected from lower alkyl group, lower alkenyl group, lower alkoxy group, lower alkoxycarbonyl group, lower alkylcarbonyl group, lower cycloalkyl group and aryl group that may be have a substituent or R₂ and R₃ may be combined to form ═CH₂ or ═O; and
R₄ and R₅ may be the same or different and each represents a hydrogen atom, a halogen atom or a group selected from lower alkyl group, lower alkenyl group, lower alkoxy group, lower alkoxycarbonyl group, lower alkylcarbonyl group, lower cycloalkyl group and aryl group that may be have a substituent or R₄ and R₅ may be combined to form ═CH₂ or ═O.

2. The nonaqueous secondary battery according to claim 1, wherein the compound of the general formula (I) is contained in the nonaqueous electrolyte solution at a proportion of 1% by volume to 60% by volume.

3. The nonaqueous secondary battery according to claim 1, wherein the compound of the general formula (I) is a compound that produces inert gas containing CO₂ or CO as a main component when heated at a temperature higher than its decomposition temperature.

4. The nonaqueous secondary battery according to claim 1, wherein the compound of the general formula (I) is a compound having a decomposition temperature of 120° C. to 250° C.

5. The nonaqueous secondary battery according to claim 1, wherein the lower alkyl group, the lower alkenyl group and the lower alkoxy group are alkyl, alkenyl and alkoxy having 1 to 6 carbon atoms, and the lower cycloalkyl group is cycloalkyl having 3 to 6 carbon atoms.

6. The nonaqueous secondary battery according to claim 1, wherein the compound of the general formula (I) is a compound formed of a combination of R₁ selected from a hydrogen atom and lower alkyl group, R₂ and R₃ selected from lower alkyl group, and R₄ and R₅ selected from a hydrogen atom and ═O combined.

7. A flame retardant for a nonaqueous secondary battery, comprising a cyclic compound having, in the molecule, a functional group having an ester bond to which a nitrogen atom is attached, in which is the general formula (I): 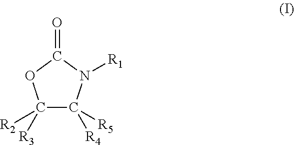
wherein R₁ is a hydrogen atom or, a group selected from lower alkyl group, lower alkenyl group, lower alkoxy group, lower alkoxycarbonyl group, lower alkylcarbonyl group, lower cycloalkyl group and aryl group that may be have a substituent;
R₂ and R₃ may be the same or different and each represents a halogen atom or, a group selected from lower alkyl group, lower alkenyl group, lower alkoxy group, lower alkoxycarbonyl group, lower alkylcarbonyl group, lower cycloalkyl group and aryl group that may be have a substituent or R₂ and R₃ may be combined to form ′CH₂ or ′O; and
R₄ and R₅ may be the same or different and each represents a hydrogen atom, a halogen atom or, a group selected from lower alkyl group, lower alkenyl group, lower alkoxy group, lower alkoxycarbonyl group, lower alkylcarbonyl group, lower cycloalkyl group and aryl group that may be have a substituent or R₄ and R₅ may be combined to form ═CH₂ or ═O.
